(19) United States
(12) Patent Application Publication
(10) Pub. No.: US 2012/0232568 A1
(43) Pub. Date: Sep. 13, 2012

(54) SURGICAL TREATMENT OF GASTRIC EMPTYING DISORDERS

(75) Inventor: John A. Cox, San Clemente, CA (US)

(73) Assignee: USGI MEDICAL, INC., San Clemente, CA (US)

(21) Appl. No.: 13/480,210

(22) Filed: May 24, 2012

Related U.S. Application Data
(63) Continuation-in-part of application No. 12/409,335, filed on Mar. 23, 2009.
Application No. 12/409,335 is a continuation-in-part of application No. 11/070,863, filed on Mar. 1, 2005, now Pat. No. 8,216,252.
Application No. 11/070,863 is a continuation-in-part of application No. 10/840,950, filed on May 7, 2004.
Application No. 10/840,950 is a continuation-in-part of application No. 10/735,030, filed on Dec. 12, 2003.
Application No. 10/735,030 is a continuation-in-part of application No. 10/639,162, filed on Aug. 11, 2003, now Pat. No. 7,618,426.
(60) Provisional application No. 61/038,487, filed on Mar. 21, 2008.

(51) Int. Cl. A61B 17/11

(52) U.S. Cl. 606/153

(57) ABSTRACT

Devices and methods for surgically altering stomach tissue to change gastric emptying. Plications are formed in the stomach speed up or slow down gastric emptying, depending on the number and locations of plications used. The plications may be formed endolumenally.

### REFERENCE TO RELATED APPLICATIONS

This application is a Continuation-in-Part of U.S. patent application Ser. No. 12/409,335 filed on Mar. 23, 2009, and now pending, which claims priority to U.S. Patent Application No. 61/038,487 filed on Mar. 21, 2008.

This Application is also a Continuation-in-Part of U.S. patent application Ser. No. 11/070,863 filed on Mar. 1, 2005 and now pending, which claims priority to U.S. patent application Ser. No. 10/840,950 filed on May 7, 2004 and now pending.

This Application is also a Continuation-in-Part of U.S. patent application Ser. No. 10/735,036 filed on Dec. 12, 2003 now pending, which is a Continuation-in-Part of U.S. patent application Ser. No. 10,639,162, filed Aug. 11, 2003, now U.S. Pat. No. 7,618,426. Each of the Applications listed above is incorporated herein by reference.

### BACKGROUND OF THE INVENTION

The stomach is a muscular hollow part of the human alimentary system, and it plays a vital role in the digestive process. It serves many purposes, including the role of reservoir for food, a role in chemical and mechanical grinding/digesting of food, and as a precursor and catalyst for a wide variety of chemical and hormonal changes before, during and after a meal. The stomach has distinct anatomical regions generally know as the fundus, corpus, (body) gastric antrum and pylorus. The stomach has a sphincter muscle at both ends which serves to manage the passage of nutrients during eating and digestion.

The stomach is surrounded by parasympathetic (stimulant) and orthosympathetic (inhibitor) plexuses, which are networks of blood vessels and nerves in the anterior gastric, posterior, superior and inferior, celiac and myenteric sections of the stomach. These regulate both the secretions activity and the motor (motion) activity of stomach muscles.

Stomach functions are controlled by both the autonomic nervous system and by the various digestive system hormones. As a reservoir, the fundus and to a lesser extent, the antrum, serve to dilate during ingestion of a meal, providing the space for short-term accommodation of the food to be digested, and for partially digested food.

The stomach has various states of activity, corresponding to pre-, intra- and post-meal functions. At the ingestion of a meal, the proximal stomach relaxes, creating a space for meal storage. The stomach begins the movement of food to the antrum, where it is mixed with digestive chemicals and is ground into chyme by muscular contractions. Once the antrum has milled the food, the pylorus opens reflexively, permitting passage to the duodenum. This partially-digested material is then passed through the pylorus and into the small bowel where further digestion and the absorption of nutrients takes place. In healthy humans, the amount of time it takes for the stomach to completely empty (gastric emptying time) is regulated very carefully to match the capacity of the duodenum to take on material and the body's ability to digest the nutrients.

Using a variety of methods, this “gastric emptying” time can be measured and used to determine if a patient has normal or abnormal characteristics. In normal patients, the presence of food in the stomach and later, in the small bowel, sets off a wide range of chemical responses that tell the brain when to eat, how much to eat, and when to stop eating. Further, scientist and clinicians are discovering that the presence and passage of nutrients through the alimentary system triggers a number of chemical processes that effect eating behavior, digestion, blood sugar, the autonomic nervous system, and the immune system. A “brain gut” link has been described in the literature and is the focus on a great deal of current research, especially as it relates to obesity, diabetes, hyperlipidemia, cardiovascular risk profile and dementia.

One area of intensive medical research focuses on the relationship between gastric emptying time and disease. It has been discovered that disordered gastric emptying (either too fast or too slow) is prevalent in patients with both Type 1 and Type 2 diabetes, and problems associated with gastric emptying result from and contribute to the symptoms and morbidities associated with the disease. In some patients, particularly those with recently-diagnosed type 2 diabetes, emptying may be accelerated, leading to a variety of serious clinical problems, and in others it is delayed, also leading to a variety of serious clinical problems. Problems include pain, nausea, vomiting, hypo- and hyper-glycemia, dumping syndrome, hyperlipidemia and hypertension. Current treatments are lacking and have significant limitations and side effects, and the prognosis of patients with disordered gastric emptying is poor.

Many patients with longstanding diabetes mellitus suffer from a condition known as gastroparesis, or severely delayed gastric emptying. Although diabetes is thought to be the cause, there are many patients with gastroparesis of unknown origin. In total, up to 75% of diabetic patients suffer from some degree of gastroparesis. Gastroparesis results from and also contributes to poor glycemic control thereby creating a cycle that increases the morbidity associated with diabetes. Patients with delayed gastric emptying could benefit from interventions that increase gastric emptying speed. Similarly, patients that suffer from accelerated emptying could benefit from therapeutic interventions that slow down gastric emptying. In either case, beyond just alleviating the immediate physical symptoms of disordered gastric emptying, therapeutic modulation of emptying can improve glycemic control and increase insulin sensitivity, thereby lessening the problems associated with diabetes. In some studies, when gastric emptying is normalized, insulin requirements post-meal have been shown to lessen.

There is a strong relationship between gastric emptying time and appetite, as well as emptying time and food intake. Gastric distension from the swallowed food, as well as nutrient stimulation of alimentary tract receptors and the release of gut peptides have a strong influence on meal size (satiation) and the amount of time before hunger returns (satiety). The effect of gastric emptying speed on obesity is just now being understood, but it is clear that the rate of gastric emptying can have an effect on a patient's body weight. Some researchers have proposed that speeding up gastric emptying time can trigger earlier responses to a meal, thus lessening food intake, while others propose that delaying or prolonging the total emptying time of a meal might reduce hunger between meals.

The mechanisms of action of these effects are not completely understood, but a leading hypothesis is that the rapid entry of partially-digested nutrients into the distal gut causes the release of GLP-1 (glucagon-like peptide 1) and peptide YY, both of which have a role in appetite and energy intake. Further, prolonging total emptying time may enhance this effect by prolonging the release of these peptides, while delaying the elevation of ghrelin and other triggers of hunger and eating behavior.

### SUMMARY OF THE INVENTION

Interventions that normalize gastric emptying can alleviate symptoms and provide a treatment for diabetes, both Type 1 and Type 2, and other conditions. For treatment of patients with accelerated gastric emptying, plications can be used in a variety of ways to slow gastric emptying. Plication of the corpus and/or antrum may create a valve or other obstacle to the rapid passage of food (i.e. speed bump). Plication of the corpus and/or antrum may be used to make normal motor function (peristalsis) inefficient or slower, thus slowing the propulsion of food to the small bowel. Plicating the pylorus may be used to tighten this valve and/or limit its opening diameter, thus slowing the passage of food.

Slowing gastric emptying by surgery of the stomach may provide an effective treatment for disorders associated with accelerated gastric emptying. By slowing emptying, release of gut hormones may be delayed, thereby prolonging satiety. This can cause weight loss by lengthening the time between meals. Surgically slowing emptying can provide a treatment for postparandial hypotension dumping syndrome. It may also improve glycemic control, providing a treatment for diabetes or its symptoms.

As a treatment for delayed gastric emptying and/or gastroparesis, plications can be used to accelerate emptying. Plication of the fundus may limit the stomach's function as a reservoir of food, or shrink its volume, thus forcing food to move to the distal stomach faster. Plicating the fundus and/or the corpus may prevent storage of food in the proximal stomach and thereby speed its delivery to the antrum. It may also propel food faster in to the duodenum.

Surgery of the stomach that speeds up gastric emptying may be a treatment for disorders associated with delayed gastric emptying, or gastroparesis. By speeding emptying, the symptoms of gastroparesis, including nausea, vomiting and pain, may be reduced. Surgically speeding emptying may also initiate gut hormone response earlier, thus triggering fullness faster and limiting meal size. It may also increase the level of certain gut hormones that optimize/improve glycemic control, thereby treating some of the symptoms of diabetes.

By speeding emptying, the level of certain gut hormones that improve insulin resistance may be increased, thereby treating diabetes. Limiting time that food stays in the stomach may reduce the stomach's ability to contribute to the digestive process, thereby moving undigested or partially digested nutrients into the distal gut. This can trigger the metabolic benefits of a gastric bypass without intestinal reconfiguration. It has been shown that gastric bypass has metabolic effects that precede the weight loss normally associated with the procedure.

Plicating the stomach may speed the emptying of some of the nutrients into the small bowel, and prolong the total emptying cycle at the same time. In combination, this could provide a treatment for obesity and diabetes. Plicating the fundus and/or body and/or antrum and/or pylorus may speed the arrival of nutrients to the small bowel. Plicating these same areas may make the digestions process inefficient, therefore making total emptying time longer.

Early release of gut hormones may limit meal size and thus caloric intake, while the presence of food in the antrum for longer periods (longer total emptying time) may delay the release of hormones that promote hunger, therefore lengthening the time between meals. Modulating and prolonging gastric emptying may reverse the negative cycle of glucose insensitivity associated with diabetes, reduce its symptoms, and or lessen the disease itself.

### BRIEF DESCRIPTION OF THE DRAWINGS

FIG. 1 is an illustration of an endolumenal system advanced endolumenally into a stomach.

FIG. 2 is an exploded view of the tissue manipulation assembly and the tissue anchor assembly delivery device shown in the system of FIG. 1.

FIG. 3 is an exploded view of a tissue anchor assembly delivery device shown in FIG. 2.

FIGS. 4A through 4C are enlarged side views of the tissue manipulation assembly and helical tissue engagement instrument of the system shown in FIG. 1.

FIG. 5 is a schematic representation of a tissue anchor assembly included in the tissue anchor assembly delivery device shown in FIG. 3.

FIG. 6 is a schematic representation of the tissue anchor assembly of FIG. 5 securing a tissue fold.

### DETAILED DESCRIPTION

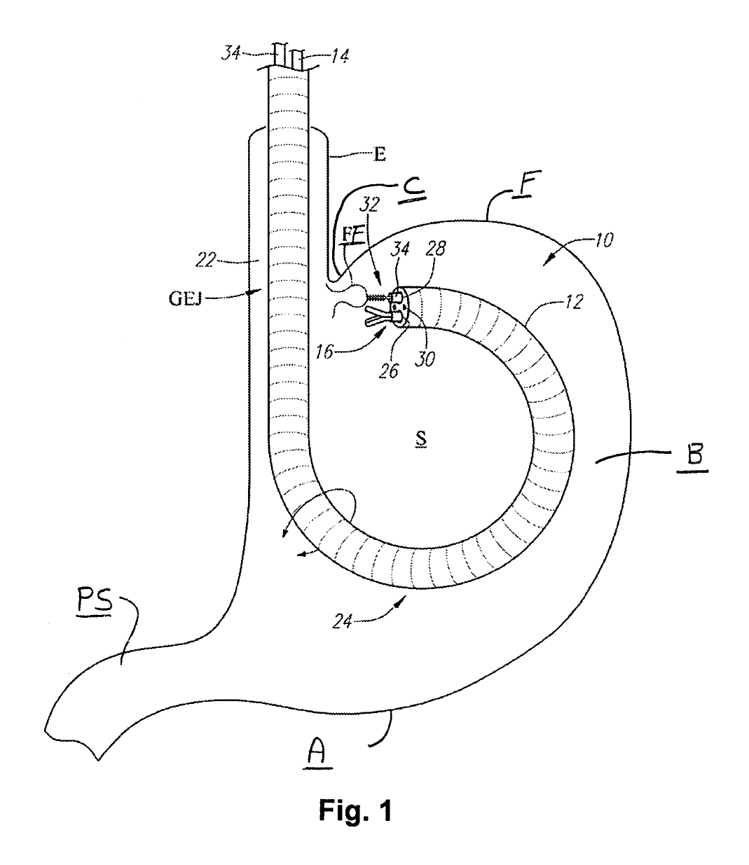

The anatomy of the stomach can be divided into different segments on the basis of the mucosal cell types and/or in relation to external anatomical boundaries. As shown in FIG. 1, the cardiac segment C is immediately subjacent to the gastroesophageal junction (GEJ) and is a transition zone of the esophageal squamous epithelium into the gastric mucosa. The fundus F is the portion of the stomach that extends above the gastroesophageal junction. The body B or corpus of the stomach extends from the fundus F to the incisura angularis on the lesser curvature of the stomach. The majority of parietal acid forming cells are present in this segment. The fundus F and the body B function as the main reservoir of ingested food. The antrum A extends from the lower border of the body B to the pyloric sphincter PS. The majority of gastrin producing or G-cells are present in the antral mucosa.

The gastrointestinal lumen, including the stomach, includes four tissue layers. The mucosa layer is the top tissue layer followed by connective tissue, the muscularis layer and the serosa layer. When plicating from the peritoneal side of the GI tract, it is easier to gain access to the serosal layer. In endolumenal approaches to surgery, only the mucosa layer is visible via an endoscope. The muscularis and serosal layers are difficult to access because they are only loosely adhered to the mucosal layer. To create a durable tissue fold or plication with suture and anchors, it is preferable to have serosa to serosa contact in the tissue fold. The mucosa and connective tissue layers typically do not heal together in a way that can sustain the tensile loads imposed by normal movement of the stomach wall during ingestion and processing of food. Folding the serosal layers with serosa-to-serosa contact allows the tissue to heal together and form a durable tissue fold, plication, or elongated invagination.

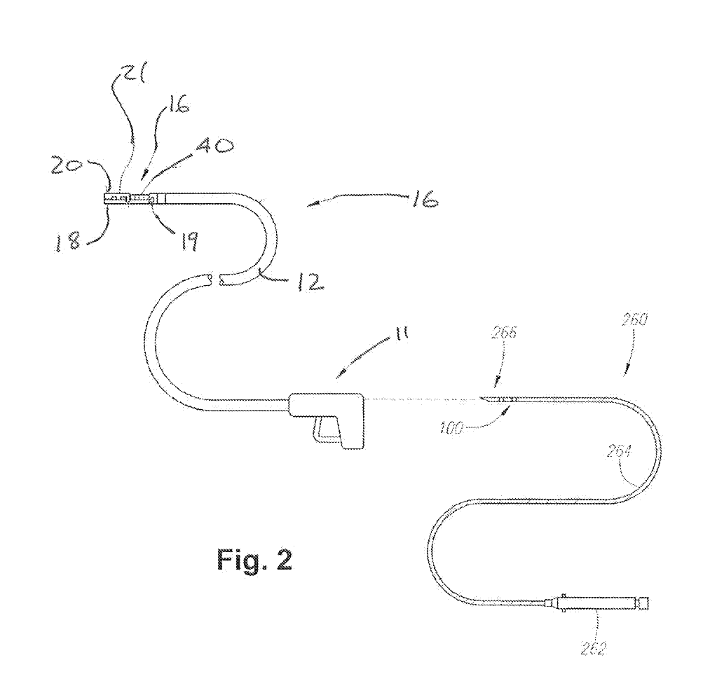

Turning now to FIGS. 1 and 2, an endolumenal system 10 includes an endoscopic body 12 having a covering 22 and a steerable distal portion 24. The endoscopic body 12 may have at least first and second lumens 26, 28, respectively. Additional lumens may be provided through the endoscopic body 12, such as a visualization lumen 30, through which an endoscope may be positioned to provide visualization. Alternatively, an imager such as a CCD imager or optical fibers may be provided in lumen 30 to provide visualization. An optional thin wall sheath may be disposed through the patient's mouth, esophagus E, and possibly past the gastroesophageal junction GEJ into the stomach S.

Referring still to FIGS. 1 and 2, the endolumenal system includes a tissue manipulation assembly 16 and a tissue anchor deployment assembly 260. The tissue manipulation assembly 16 includes a flexible catheter or tubular body 12 which is sufficiently flexible for advancement into a body lumen, e.g., transorally, percutaneously, laparoscopically, etc. The tubular body 12 is torqueable through various methods, e.g., utilizing a braided tubular construction, such that when a handle 11 is manipulated and/or rotated by a practitioner from outside the patient's body, the longitudinal and/or torquing force is transmitted along the body 12 such that the distal end of the body 12 is advanced, withdrawn, or rotated in a corresponding manner. Jaws 18 and 20 are attached to the front end of the body 12, optionally at a pivot joint connection 19.

A launch tube 40 extends through the body 12 may be pivotally attached to the upper jaw. The front end of the launch tube may be designed to change from straight into a curved or arcuate shape when the launch tube is advanced forward. When in the curved shape, the launch tube opening may be generally perpendicular to the upper jaw 20. The launch tube 40, or at least the exposed portion of the launch tube 40, may be fabricated from a highly flexible material or it may be fabricated, e.g., from Nitinol tubing material which is adapted to flex, e.g., via circumferential slots, to permit bending. Movement of the launch tube may also open and close the jaws. Using the launch tube 40 to articulate the jaws eliminates the need for a separate jaw moving mechanism.

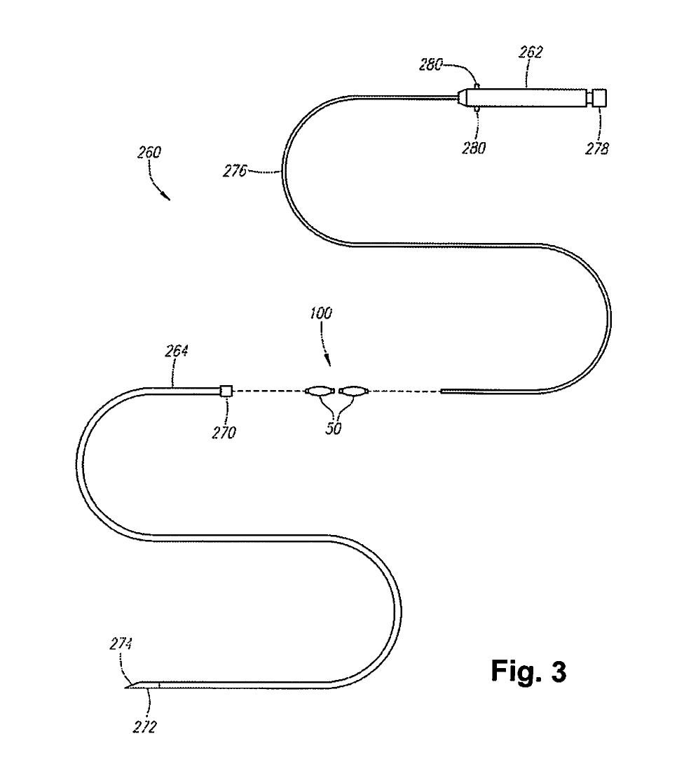

As shown in FIG. 3, the tissue anchor assembly delivery system 260 may be deployed through the tissue manipulation assembly 16 by sliding it in through the handle 11 and through the tubular body 12. Once the needle 272 has been advanced through the tissue fold FF, the first anchor assembly 100 may be deployed or ejected. The anchor assembly 100 is normally positioned within the distal portion of a tubular sheath 264. Once the anchor assembly 100 has been fully deployed from the sheath 264, the spent tissue anchor assembly delivery system 260 may be removed and replaced from the tissue manipulation assembly 16 without having to remove the tissue manipulation assembly 16 from the patient.

The sheath or catheter 264 and the housing 262 may be interconnected via an interlock 270 which may be adapted to allow for the securement as well as the rapid release of the sheath 264 from the housing 262 through any number of fastening methods, e.g., threaded connection, press-fit, releasable pin, etc.

A pusher 276 which may be a flexible wire or tube within the sheath slides within the housing 262. An actuator 278 on the housing 262 is used to slide the pusher 276 relative to the sheath 264, to push anchors out from the opening 274 at the tip of the needle 272. Needle assembly guides 280 may protrude from the housing 262 for guidance through the locking mechanism.

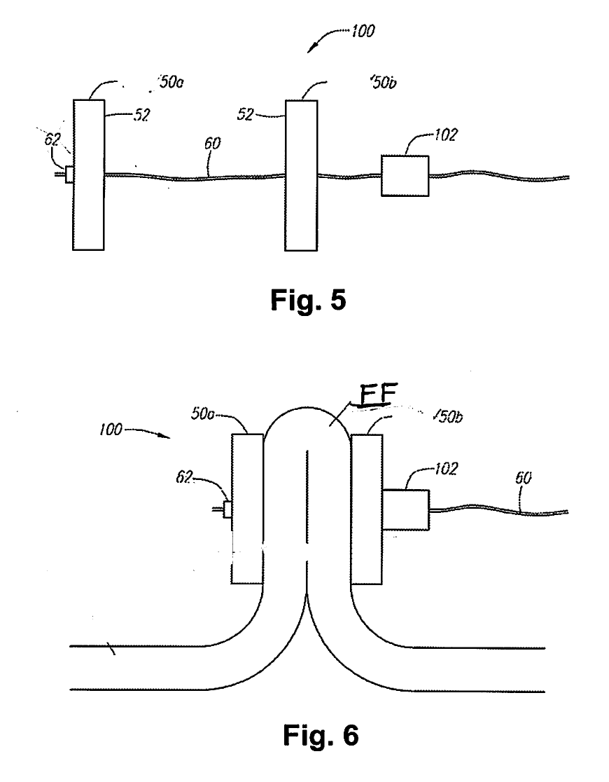

As shown in FIG. 5, typically, the tissue anchor assemblies include a pair of tissue anchors 50a and 50b, slidably attached to a suture 60. A knot 62 or other protrusion on the distal end of the suture keeps the distal anchor assembly from sliding off the end of the suture 60. The suture runs back up through the catheter 264 to the control handle 262, so that after both anchor assemblies have been deployed, the surgeon can tension the suture. A locking mechanism, such as a cinch 102, is also slidably retained on the suture 60. The cinch 102 is configured to provide a cinching force against the anchors to impart a tension force on the suture. With the suture under tension, the proximal anchor assembly 50b and the cinch 102 are pushed up against the fold FF. Accordingly, the tissue anchor assembly 100 is adapted to hold a fold of tissue, as shown in FIG. 6.

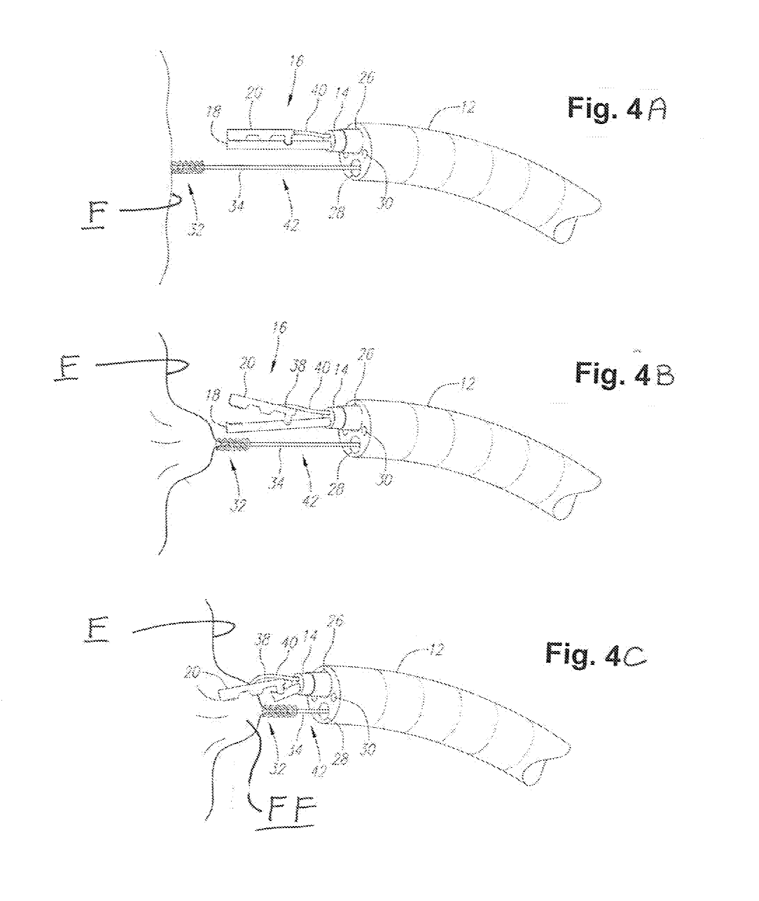

Surgery on the stomach to speed up or slow down gastric emptying may be performed as follows. The surgical site within the stomach may be visualized through the visualization lumen 30 or a separate imager. In either case, the tissue manipulation assembly 16 and the tissue engagement member 32 may be advanced distally out from the endoscopic body 12 through lumens 26, 28. The distal steerable portion 24 of the endoscopic body 12 is steered to an orientation to position the jaws to engage stomach tissue. FIG. 1 shows a tissue manipulation assembly 16 advanced through the first lumen 26 and a helical tissue engagement member 32 positioned upon a flexible shaft 34 advanced through the second lumen 28. To obtain a durable tissue fold FF, the engagement member 32 is advanced or corkscrewed into tissue, as shown in FIG. 4A. The jaws are opened, optionally by pulling launch tube 40 back as shown in FIG. 4B.

The engagement member 32 is then pulled back to draw the engaged tissue FF between the jaws 18 and 20, as shown in FIG. 4C. Once the tissue has been pulled or manipulated between the jaws, the jaws are closed, in this case by pushing the launch tube 40 forward. Movement of the launch tube may also change the angle of the jaws and the front end of the launch tube relative to the tissue.

With the tissue engaged between the jaws 18, 20, a needle assembly may be fed through the handle with the needle 272 moving out of the front end of the launch tube 40. The needle 272 pierces through the engaged tissue fold FF. The pusher is then used to push out the first anchor. The needle 272 is then pulled back through the tissue fold FF and the second anchor is deployed. The cinch and the second anchor are pushed up against the tissue fold FF, using the jaws or another instrument, to form a permanent tissue fold. Using the methods described above, permanent tissue folds or plications FF may be made in the stomach, to alter gastric emptying.

Although the methods above are described as endolumenal trans-oral methods, these same methods may be performed in other ways as well, such as trans-anally, percutaneously, laparoscopically, robotically, or even via traditional open body surgery.

Thus, novel systems and methods have been shown and described. Various changes and substitutions may of course be made without departing from the spirit and scope of the invention. The invention, therefore, should not be limited, except by the following claims and their equivalents.

1. A method for treating accelerated gastric emptying, comprising:
forming two or more plications of the corpus and/or the antrum of the stomach to create an obstacle in the stomach to the rapid passage of food through the stomach, with the obstacle slowing gastric emptying.

2. The method of claim 1 with the obstacle comprising a valve.

3. The method of claim 1 with the obstacle delaying the release of gut hormones and thereby prolonging satiety, resulting in weight loss by lengthening the time between meals.

4. A method for treating accelerated gastric emptying, comprising:
forming two or more plications of the corpus and/or the antrum of the stomach, with the plications slowing peristalsis, thus slowing the propulsion of food to the small bowel.

5. The method of claim 4 with the plications delaying the release of gut hormones and thereby prolonging satiety, resulting in weight loss by lengthening the time between meals

6. The method of claim 4 with the slowing peristalsis changing glycemic control to provide a treatment for diabetes or its symptoms.

7. A method for treating delayed gastric emptying, comprising:
forming two or more plications in tissue of the stomach to reduce the volume of the stomach and force food to move through the stomach more quickly.

8. The method of claim 7 with the plications formed in the fundus and/or the corpus to prevent storage of food in the proximal stomach and thereby speed delivery of food to the antrum.

9. The method of claim 7 with the plications reducing symptoms of gastroparesis, including nausea, vomiting and pain.

10. The method of claim 7 with the plications initiating gut hormone response earlier, thus triggering fullness faster and limiting meal size.

11. The method of claim 7 with the plications speeding emptying and increasing the level of gut hormones that improve glycemic control, thereby providing a treatment for symptoms of diabetes.

12. The method of claim 7 with the plications limiting the amount of time food stays in the stomach which reduces the stomach's ability to contribute to the digestive process, and moving undigested or partially digested food into the distal gut, triggering the metabolic benefits of a gastric bypass without intestinal reconfiguration.

13. The method of claim 7 with plications formed in the fundus and/or body and/or antrum and/or pylorus to speed the arrival of nutrients to the small bowel.

14. The method of claim 7 with the plications resulting in early release of gut hormones to limit meal size and caloric intake, with the presence of food in the antrum for longer periods of time delaying release of hormones that promote hunger, therefore lengthening the time between meals.

15. A method for treating a gastric emptying disorder, comprising:
a) advancing a delivery catheter through a patient's mouth and esophagus and into the patient's stomach;
b) forming a tissue fold in the tissue of the stomach;
c) passing the needle through the tissue fold;
d) deploying a first tissue anchor assembly from the needle on a distal side of the tissue fold;
e) withdrawing the needle back through the tissue fold;
f) deploying a second tissue anchor assembly from the needle on a proximal side of the tissue fold, with the second tissue anchor assembly linked to the first tissue anchor assembly by a suture;
g) securing the second tissue anchor assembly in place to form a substantially permanent plication in the stomach;
h) with the plication altering the gastric emptying.

16. The method of claim 15 further comprising forming additional plications in the stomach by repeating at least steps b-g.

17. The method of claim 15 with the plications formed in the fundus.

18. The method of claim 15 with the plications formed in the antrum.

19. The method of claim 15 with the plications formed in the pylorus.

20. The method of claim 15 with the plications forming an obstacle to movement of food through the stomach.

21. The method of claim 1 with the obstacle delaying the release of gut hormones and thereby preventing post-parandial hypotension.

22. The method of claim 7 with the plications causing stretch receptors in the stomach to fire upon ingestion of food, thus creating early feelings of fullness and limiting meal size.
